(19) United States
(12) Patent Application Publication
(10) Pub. No.: US 2005/0272643 A1
(43) Pub. Date: Dec. 8, 2005

(54) Removal of serum albumin from human serum or plasma

(75) Inventors: Henry Brzeski, Johnstown, PA (US); Stephen Russell, Johnstown, PA (US)

Correspondence Address: STEPTOE & JOHNSON LLP;ATTORNEYS AT LAW, 1330 Connecticut Avenue, NW, Washington, DC, 20036-1795, US

(73) Assignee: Windber Research Institute, Windber, PA (US)

(21) Appl. No.: 10/862,879

(22) Filed: Jun. 8, 2004

(51) Int. Cl. A61K 38/38; C07K 14/765

(52) U.S. Cl. 514/8; 530/363

(57) ABSTRACT

A method of removing serum albumin from blood serum or plasma includes contacting the blood serum or plasma with a sugar-binding protein such as a lectin (e.g., Concanavalin A or wheat germ agglutinin) immobilized on an insoluble support such as a porous bead (e.g., agarose). The method may be practiced by immobilizing a sugar-binding protein on an insoluble support, preparing blood serum or plasma for contacting with the insoluble support having the sugar-binding protein immobilized thereon, contacting the prepared blood serum or plasma with the insoluble support having the sugar-binding protein immobilized thereon to absorb glycosylated proteins from the blood serum or plasma leaving an unbound fraction containing serum albumin, and differentially eluting glycosylated proteins with different sugars from the insoluble support contacted with the blood serum or plasma.

### BACKGROUND OF THE INVENTION

1. Field of the Invention

The present invention relates to the removal of serum albumin from blood serum or plasma prior to analysis, such as two dimensional gel electrophoresis, of human serum/plasma.

2. Description of the Prior Art

In isoelectric focusing, molecules with a net charge, such as proteins, move toward one electrode or another when placed in a specially designed gel containing ampholytes and then placed in an electric field. The greater the net charge, the further the molecules will move from the centre of the gel. In SDS (sodium dodecyl sulfate) polyacrylamide gel electrophoresis, the molecular separation is based on the size of the protein since the separation is carried out in a gel which acts like a molecular sieve. The gels are commonly made from polyacrylamide which is chemically inert. The pore size of the gel can be carefully controlled.

In two-dimensional gel electrophoresis, proteins are first separated according to charge in polyacrylamide gel by isoelectric focusing. Isoelectric focusing electrophoretically separates proteins on the basis of their relative content of positively and negatively charged groups. The gel is then rotated by 90 degrees and the proteins electrophoresed into gel containing SDS, which further separates the proteins by mass. A 2-D map of protein spots is thus created which can contain several thousand resolved species. Individual spots are then cut from the gel and treated with proteases to produce a set of peptides characteristic of that protein. Mass spectrometry is then used to produce a peptide mass fingerprint of that protein which is compared to a database of predicted fingerprints.

The major protein found in human serum/plasma is serum albumin. Serum albumin, among other things, serves to maintain the osmotic pressure of the blood. Serum is prepared by collecting blood without an anticoagulant, allowing it to clot and this is followed by centrifugation of blood after coagulation. The supernatant (serum) is a yellow watery fluid containing no cells. The pellet (clot) contains erythrocytes, leukocytes and platelets. Serum albumin constitutes about 60 to 80% of all protein present in human serum/plasma.

Electrophoresis of human serum/plasma is often used as an indication of the physiological state of an individual. Unfortunately, effective separation of the serum/plasma proteins is prevented by the excessive amounts of serum albumin present.

A current procedure for removal of serum albumin is based on the use of a blue dye linked to an insoluble support. This dye has an affinity for albumin (and other proteins). If serum/plasma is passed over columns packed with the dye, then serum albumin should bind to the column while other proteins pass straight through the column. The chemical kinetics are such that not all the serum albumin binds to the column and, under normal conditions, the unbound fraction contains residual serum albumin.

To illustrate the above, kits based on Cibracron blue dye are commercially available from ProMetic Life Sciences Inc., Bio-Rad Laboratories, Millipore Corporation, Pierce Biotechnology and Applied Biosyatems. Removal of serum albumin using Cibacron blue suffers from a lack of specificity. The Cibacron blue dye binds many proteins other than albumin, such as lipoproteins, blood coagulation factors, etc.

Another method for the removal of serum albumin from human serum/plasma is based on antibodies bound to an insoluble support that are specific for albumin and perhaps other proteins. This procedure does not appear to be quantitative and some serum albumin, and other proteins that should bind to the antibodies, are found in the eluate from the column.

The amount of sugar combined with proteins is a refection of metabolic state and may provide important diagnostic information concerning health status. The majority of proteins in serum/plasma are glycosylated, i.e., they have large polymeric chains of different types of sugar attached to them. Thus, analysis of differences in glycosylation is difficult because of the heterogeneous nature of the samples.

### SUMMARY OF THE INVENTION

The present invention has two goals: (a) to remove serum albumin from human serum/plasma prior to electrophoresis, especially on two-dimensional gels, and (b) to resolve serum/plasma proteins based on their extent of glycosylation. The invention works on the principal that serum albumin is not highly glycosylated and that the remaining proteins have differential binding capacity.

The present invention uses a sugar binding protein bound to an insoluble support. The sugar binding protein is lectin. Lectins have the ability to bind particular sugars and different lectins can bind two or three structurally related sugars. The insoluble support is preferably agarose. The presently preferred process is a batch procedure for protein absorption. However, a continuous column procedure is envisioned.

The majority of proteins in serum/plasma are bound to lectin beads or the like. Then, the differential binding capacity is taken advantage of to elute different proteins by using different sugars to analyze differential glycosylation.

The lectin carries out the binding. The insoluble support provides a mechanism to quickly separate bound from unbound proteins.

The invention provides a reliable method for serum/plasma removal unlike currently available methods. The invention also provides a more effective method than current methods.

Other exemplary embodiments and advantages of the present invention may be ascertained by reviewing the present disclosure.

### DESCRIPTION OF THE PREFERRED EMBODIMENTS

In accordance with the present invention, sugar binding protein is immobilized on an insoluble support. The immobilized sugar binding protein is then allowed to bind to a glycosylated protein in blood serum or plasma. The unbound residual material (serum albumin) is removed by subsequent washing. The bound glycolated proteins are displaced from the immobilized sugar binding proteins by any suitable means.

The sugar binding protein is preferably a lectin. The term “lectin” as used herein refers to a sugar-binding protein of non-immune origin that agglutinates cells or precipitates glycoconjugates. The lectin molecule contains at least two sugar-binding sites; sugar-binding proteins with a single site will not agglutinate or precipitate structures that contain sugar residues, so are not typically classified as lectins. The specificity of a lectin is usually defined by the monosaccharides or oligosaccharides that are best at inhibiting the agglutination or precipitation the lectin causes. Lectins occur in many types of organism; they may be soluble or membrane-bound; they may be glycoproteins. Sugar-specific enzymes, transport proteins and toxins may qualify as lectins if they have multiple sugar binding sites.

Mannose binding lectins include Concanavalin A (Con A) (Canavalia ensiformis); lentil lectin (LCH) (Lens culinaris) and snowdrop lectin (GNA) (Galanthus nivalis). Sialic acid/N-acetylglucosamine binding lectins include wheat germ agglutinin (WGA) (Triticum vulgaris); elderberry lectin (SNA) (Sambucus nigra) and maackia amurensis lectin (MAL) (Maackia amurensis). Galactose/N-acetylgalactosamine binding lectins include ricinus communis agglutinin (RCA) (Ricinus communis); coral tree lectin (ECL) (Erythrina cristagalli); peanut agglutinin (PNA) (Arachis hypogaea); jacalin (AIL) (Artocarpus integrifolia); and hairy vetch lectin (VVL) (Vicia villosa). Fucose binding lectins include ulex europaeus agglutinin (UEA) (Ulex europaeus) and aleuria aurantia lectin (AAL) (Aleuria aurantia). The preferred lectins are wheat germ agglutinin and Concanavalin A (Con A).

Wheat germ agglutinin is a 36,000 molecular weight protein consisting of two identical subunits. Wheat germ agglutinin contains a group of closely related isolectins, with an isoelectric point about pH 9. The receptor sugar for wheat germ agglutinin is N-acetylglucosamine, with preferential binding to dimers and trimers of this sugar. Wheat germ agglutinin can bind oligosaccharides containing terminal N-acetylglucosamine or chitobiose, structures which are common to many serum and membrane glycoproteins. Bacterial cell wall peptidoglycans, chitin, cartilage glycosaminoglycans and glycolipids can also bind wheat germ agglutinin. Native wheat germ agglutinin has also been reported to interact with some glycoproteins via sialic acid (N-acetyl neuraminic acid) residues.

Concanavalin A has broad applicability primarily because it recognizes a commonly occurring sugar structure, a-linked mannose. Since a wide variety of serum and membrane glycoproteins have a “core oligosaccharide” structure which includes α-linked mannose residues, many glycoproteins can be examined or purified with Concanavalin A and its conjugates. At neutral and alkaline pH, Concanavalin A exists as a tetramer of four identical subunits of approximately 26,000 daltons each. Below pH 5.6, Concanavalin A dissociates into active dimers of 52,000 daltons. “Native” Concanavalin A is a mixture of several forms of the lectin. Concanavalin A has an isoelectric point of about pH 5. Concanavalin A has also been reported to interact with some glycoproteins via glucose residues

The insoluble support is typically a porous bead. Small molecules can enter the pores in the beads whereas larger or more elongated molecules cannot. A preferred insoluble support is agarose (e.g., Sepharose®, a chemically cross-linked agarose). Agarose is a linear galactan created by purifying agar. When it is heated and cooled, it forms a gel that is useable as a support for many types of electrophoresis. A typical gel is about 1 to 6% agarose. Agarose is more porous than acrylamide and is sold in different grades; the lower its sulfate content, the more highly purified it is.

The invention may be practiced, for example, using wheat germ agglutinin bound to agarose (Sigma-Aldrich Product No. L1394) or Concanavalin A immobilized on Sepharose® 4B (Sigma-Aldrich Product No. 27700). Similar products are available from Amersham Biosciences and Vector Laboratories.

The following method of bead preparation, plasma (or serum) preparation, absorption of protein to immobilize lectin and elution of glycosalated proteins is illustrative of the invention:

Bead Preparation

1. Disperse 200 μl of lectin bead suspension per tube (i.e., assuming solution is approximately 100 μl bead+100 μl solution);
2. Centrifuge at 10 g for 1 minute to pellet beads;
3. Remove and discard all buffer;
4. Add 500 μl of 1× equilibration buffer² and shake;
5. Repeat steps 2 to 4 three times to prepare beads;
6. Centrifuge at 10 g for 1 minute to pellet beads; and
7. Remove and discard all buffer.
   Plasma Preparation
8. Add 50 μl of plasma to four separate tubes. Add 100 μl of 2× equilibration buffer¹ to each tube. Add 49 μl of deionized water to the respective tube. Mix well. Add 1 μl of 5% SDS and mix well.
   Absorption of Protein to Immobilized Lectin
9. Add 200 μl of diluted plasma (step 8) to prepared beads (step 7);
10. Place tube on its side on a rotary shaking platform and shake gently (5 minutes for 1 st binding step, 2 minutes for subsequent washing steps);
11. Centrifuge at 10 g for 1 minute to pellet beads;
12. Remove all buffer/plasma with a gel loading tip and save in tube labeled “unbound;”
13. Add 500 μl of 1× equilibration buffer² to the beads and shake for 2 minutes;
14. Repeat steps 11-13 three times to wash beads and pool wash fractions;
15. Centrifuge at 10 g for 1 minute to pellet beads; and
16. Remove all buffer/plasma with a gel loading tip and save in tube labeled “unbound.”
   Elution of Glycosylated Proteins
17. Add 200 μ of 1× elution buffer³ to the beads in the tube;
18. Place tube on its side on the rotary shaking platform and shake gently (5 minutes for 1st elution step, 2 minutes for subsequent washing steps);
19. Centrifuge at 10 g for 1 minute to pellet beads; and
20. Remove all buffer with a gel loading tip and save in tube labeled “bound.”

¹ 2× Equilibration Buffer (0.1 M sodium phosphate, pH 7.0/0.4 M NaCl)

| | |
|---|---|
| Na₂HPO₄ (anhydrous) | 3.549 g |
| NaCl | 5,843 g |

² 1× Equilibration Buffer
10 mL 2× Equilibration Buffer
10 mL de-ionized water
   ³ 1× Elution Buffer-N-acetylglucosamine
1.0 mL N-acetyglucosamine
1.0 mL 2× Equilibration Buffer
   1× Elution Buffer N-acetylneuraminic acid
1347 ul N-acetylneuraminic acid
1347 ul 2× Equilibration Buffer

In the above illustrative example, the “unbound” fraction contains the serum albumin. The “bound” fraction contains the glycosylated proteins. The lectin bead suspension can be formed from a single lectin or a plurality of lectins. The capacity of the lectins to bind multiple sugars can be taken advantage of to elute and separate different proteins. The “bound” fraction can be subjected to electrophoresis, such as two dimensional gel electrophoresis, or other analysis.

It is noted that the foregoing example has been provided merely for the purpose of explanation and is in no way to be construed as limiting of the present invention. While the present invention has been described with reference to certain preferred embodiments, it is understood that the words which have been used herein are words of description and illustration, rather than words of limitation. Changes may be made without departing from the scope and spirit of the present invention in its aspects. Although the present invention has been described herein with reference to particular materials and embodiments, the present invention is not intended to be limited to the particulars disclosed herein; rather, the present invention extends to all functionally equivalent methods and uses.

1. A method of removing serum albumin from blood serum or plasma, comprising contacting the blood serum or plasma with a sugar-binding protein immobilized on an insoluble support.

2. The method of claim 1, wherein the sugar-binding protein is a lectin.

3. The method of claim 2, wherein the lectin is a mannose and/or glucose binding lectin.

4. The method of claim 3, wherein the mannose and/or glucose binding lectin is Concanavalin A.

5. The method of claim 2, wherein the lectin is a N-acetylglucosamine and/or N-acetyl neuraminic acid binding lectin.

6. The method of claim 5, wherein the N-acetylglucosamine and/or N-acetyl neuraminic acid binding lectin is wheat germ agglutinin.

7. The method of claim 1, wherein the insoluble support is a porous bead.

8. The method of claim 7, wherein the porous bead is agarose.

9. The method of claim 1, wherein the insoluble support is subjected to elution after contact with the blood serum or plasma.

10. A method of removing serum albumin from blood serum or plasma, comprising immobilizing a lectin on an insoluble support, preparing blood serum or plasma for contacting with the insoluble support having the lectin immobilized thereon, contacting the prepared blood serum or plasma with the insoluble support having the lectin immobilized thereon to absorb glycosylated proteins from the blood serum or plasma, and eluting glycosylated proteins from the insoluble support contacted with the blood serum or plasma.

11. The method of claim 10, wherein the blood serum or plasma is blood serum.

12. The method of claim 10, wherein the blood serum or plasma is blood plasma.

13. The method of claim 10, wherein the lectin is Concanavalin A or wheat germ agglutinin.

14. The method of claim 10, wherein the insoluble support is agarose.

15. A method of removing serum albumin from blood serum or plasma, comprising contacting the blood serum or plasma with a lectin immobilized on an insoluble porous bead support.

16. The method of claim 15, wherein the lectin is Concanavalin A or wheat germ agglutinin.

17. The method of claim 15, wherein the insoluble porous bead support is agarose.

18. A method of removing serum albumin from blood serum or plasma, comprising contacting the blood serum or plasma with a lectin immobilized on an insoluble agarose porous bead support.

19. A method of treating human blood serum or plasma, comprising contacting the human blood serum or plasma with a sugar-binding protein immobilized on an insoluble support and subjecting the bound fraction to electrophoresis.

20. The method of claim 19, wherein the sugar binding protein is a lectin and the insoluble support is agarose.

21. The method of claim 19, wherein electrophoresis is two dimensional gel electrophoresis.

22. A method of removing serum albumin from blood serum or plasma, comprising immobilizing a lectin on an insoluble support, preparing blood serum or plasma for contacting with the insoluble support having the lectin immobilized thereon, contacting the prepared blood serum or plasma with the insoluble support having the lectin immobilized thereon to absorb glycosylated proteins from the blood serum or plasma and leave an unbound fraction containing serum albumin, and eluting glycosylated proteins from the insoluble support contacted with the blood serum or plasma.

23. The method of claim 22, a plurality of different lectins are immobilized on a plurality of insoluble supports and glycosylated proteins are differentially eluted from the insoluble supports.
